(19) United States
(12) Patent Application Publication
(10) Pub. No.: US 2026/0174710 A1
(43) Pub. Date: Jun. 25, 2026

(54) Compositions and Methods for Treating Arthritis

(71) Applicant: YUJIAN International Biotechnology, Inc., Hsin-Chu (TW)

(72) Inventors: Yao-Jen TU, Hsin-Chu (TW); Yu-Shan LIU, Hsin-Chu (TW); Chih-Yuan CHENG, Hsin-Chu (TW); Yi-Ling YANG, Hsin-Chu (TW); Chin-Chang YANG, Hsin-Chu (TW)

(21) Appl. No.: 19/086,145

(22) Filed: Mar. 21, 2025

(30) Foreign Application Priority Data
Dec. 20, 2024 (TW) 113150055

(51) Int. Cl. A61K 31/122; A61K 31/7028; A61K 31/728; A61K 33/04; A61K 33/06; A61K 33/18; A61K 33/24; A61K 33/26; A61K 33/30; A61K 33/32; A61K 33/34; A61K 33/42; A61K 36/02; A61K 36/185; A61K 38/39; A61P 19/02

(52) U.S. Cl. CPC A61K 31/122; A61K 31/7028; A61K 31/728; A61K 33/04; A61K 33/06; A61K 33/18; A61K 33/24; A61K 33/26; A61K 33/30; A61K 33/32; A61K 33/34; A61K 33/42; A61K 36/02; A61K 36/185; A61K 38/39; A61P 19/02

(57) ABSTRACT

The present disclosure concerns a composition for treating arthritis. The composition comprises astaxanthin, a seaweed calcium composition, collagen, calcium fructoborate, a Terminalia chebula extract, and a palm fruit extract. The present disclosure is also related to a method for treating arthritis, comprising administering a composition of the present disclosure to a subject to alleviate a symptom of arthritis.

### CROSS-REFERENCE TO RELATED APPLICATIONS

This application claims priority to co-pending Taiwan patent application No. 113150055, filed on Dec. 20, 2024, titled “Compositions and Methods for Treating Arthritis,” which is incorporated herein by reference for its entirety.

### TECHNICAL FIELD

The present disclosure is related to compositions and methods for alleviating and treating symptoms of arthritis, particularly to compositions and methods for relieving and treating symptoms of degenerative arthritis.

### BACKGROUND OF THE INVENTION

Arthritis is generally referred to as an illness having symptoms such as joint inflammation, causing swelling, stiffness, pain, and limiting movement in joints. The causes of arthritis can vary and commonly include age, overuse, and diseases (for example, infections or auto-immune diseases happening in joints). The most common types of arthritis are degenerative arthritis (or osteoarthritis) and rheumatoid arthritis, and among them, degenerative arthritis is particularly related to age factors and joint overuse. As advanced and developed countries gradually enter an aging society, and with the rising awareness of obesity and exercise leading to an increase in cases of joint overuse, the demand for relief and treatment of degenerative arthritis in this field is also growing.

### BRIEF SUMMARY OF THE INVENTION

The present disclosure is related to a composition for treating arthritis, comprising, in every 1000 mg, 1 to 10 mg of astaxanthin; 30 to 200 mg of seaweed calcium composition, comprising at least 10 wt % of calcium; 5 to 100 mg of collagen; 20 to 150 mg of hyaluronic acid; 5 to 50 mg of calcium fructoborate; 100 to 500 mg of Terminalia chebula extract; and 100 to 500 mg of palm fruit extract.

The present disclosure is also related to a method of treating arthritis, comprising administering the composition of the present disclosure to a subject.

### BRIEF DESCRIPTION OF THE SEVERAL VIEW OF THE DRAWING

FIG. 1 is a graphic presentation showing the weight-change rate of the control rats and the experimental rats during the period of the experiments. The weight-change rate was calculated using the formula: ((current weight-initial weight)/initial weight)×100%. The values were presented in mean±SEM; wherein *p<0.05 and **p<0.01.

FIG. 2 is a graphic presentation showing the water intake of the control rats and the experimental rats during the period of the experiments. The water intake was calculated using the formula: (Total volume of water intake of a given week)/(Number of mice in the cage). The values were presented in mean±SEM; wherein *p<0.05 and **p<0.01.

FIG. 3 is a graphic presentation showing the food intake of the control rats and the experimental rats during the period of the experiments. The food intake was calculated using the formula: (Total volume of food intake of a given week)/(Number of mice in the cage). The values were presented in mean±SEM; wherein *p<0.05 and **p<0.01.

FIG. 4 is a graphic presentation showing the width ratio of the right knee to the left knee of the control rats and the experimental rats during the period of the experiments. The values were presented in mean±SEM; wherein *p<0.05 and **p<0.01.

FIG. 5A is a photo of a histochemistry staining result, showing the right joint of a rat of the control group observed at the time when the surgery was just completed. The black arrow points to the articular cartilage lesion area.

FIG. 5B is a photo of a histochemistry staining result, showing the right joint of a rat of the experimental group observed at the time when the surgery was just completed. The black arrow points to the articular cartilage lesion area.

FIG. 5C is a photo of a histochemistry staining result, showing the right joint of a rat of the control group observed one month after surgery. The black arrow points to the articular cartilage lesion area.

FIG. 5D is a photo of a histochemistry staining result, showing the right joint of a rat of the experimental group observed one month after surgery. The black arrow points to the articular cartilage lesion area.

FIG. 5E is a photo of a histochemistry staining result, showing the right joint of a rat of the control group observed two months after surgery. The black arrow points to the articular cartilage lesion area.

FIG. 5F is a photo of a histochemistry staining result, showing the right joint of a rat of the experimental group observed two months after surgery. The black arrow points to the articular cartilage lesion area.

FIG. 5G is a photo of a histochemistry staining result, showing a joint of a normal rat that did not undergo the surgery conducted to create the joint lesion.

### DETAILED DESCRIPTION OF THE INVENTION

The treatment of arthritis nowadays still primarily focuses on relieving pain and is usually performed together with adjunct therapies that manage the symptoms and reduce the frequency of joint inflammation. Clinically, there are various painkillers available for healthcare professionals to choose from to alleviate pain, such as Aspirin, Ibuprofen, Indomethacin, and Naproxen, which are non-steroidal anti-inflammatory drugs (NSAIDs). Painkillers are typically prescribed when pain occurs, but given the concerns of side effects, they shouldn't be used too often and are usually not recommended for preventing arthritis. The aforesaid adjunct therapies focus on changing lifestyles or exercises to reduce the burden on the joints. Some common approaches include controlling and maintaining a proper weight, avoiding over-exercise and ensuring sufficient rest afterward, wearing joint braces during exercise or daily activities, using heat therapy or massage to relieve muscle tension and promote blood circulation, etc. The effects of those adjunct therapies, however, are limited and dependent on patients' compliance.

Furthermore, as arthritis is becoming more and more common and draws from academic and industrial sectors, there are many health supplement products on the market that claim joint care functionalities. Many of those health supplement products advertise their inclusion of novel and scarce natural ingredients. However, even though those natural ingredients have been proven by academic research to show effects on relieving arthritis symptoms, they are usually costly, so those supplement products usually comprise only a small amount of those ingredients, making the actual effects of those products doubtful. Accordingly, the field needs novel compositions that combine a variety of active ingredients capable of alleviating arthritis at a proper ratio for synergistic effects. As a result, those kinds of compositions are expected to obtain similar or better effects while using each individual ingredient in a smaller amount.

### Compositions of the Present Disclosure

The present disclosure provides a composition for treating arthritis. As used herein, “treat,” “treating,” “treatment,” or alike, when used to refer to arthritis, means alleviating or improving symptoms of arthritis, wherein the symptoms can be which is diagnosed by a medical practitioner or is self-diagnosed by the subject who is suffering the symptoms. For example, alleviating or improving symptoms of arthritis might include, but are not limited to, alleviating joint pain, reducing joint inflammation, improving joint movement, or a combination thereof; while “alleviating,” reducing,” or “improving” is relative to a benchmark level before a subject is administered with the composition of the present disclosure, and the alleviating, reducing, or improving extent can be determined by a medical practitioner or by the subject. As used herein, “treat,” “treating,” “treatment,” or alike may also include providing preventive effects for individuals who have the disease or symptoms or are at risk of developing the disease or symptoms (e.g., recurrence).

In many embodiments, the composition of the present disclosure comprises astaxanthin, seaweed calcium composition, collagen, hyaluronic acid, calcium fructoborate, Terminalia chebula extract, and palm fruit extract. In every dosage with 1000 mg, the composition comprises 1 to 10 mg of astaxanthin; 30 to 200 mg of seaweed calcium composition; 5 to 100 mg of collagen; 20 to 150 mg of hyaluronic acid; 5 to 50 mg of calcium fructoborate; 100 to 500 mg of Terminalia chebula extract; and 100 to 500 mg of palm fruit extract. In a specific embodiment, the composition of the present disclosure comprises about 4 mg of astaxanthin; about 95 mg of seaweed calcium composition, comprising 10 to 50 wt % of calcium; about 40 mg of undenatured collagen Type II; about 80 mg of hyaluronic acid; about 25 mg of calcium fructoborate; about 250 mg of Terminalia chebula extract; and about 350 mg of palm fruit extract.

In some practical embodiments, the composition of the present disclosure consists substantially of 1 to 10 mg of astaxanthin; 30 to 200 mg of seaweed calcium composition; 5 to 100 mg of collagen; 20 to 150 mg of hyaluronic acid; 5 to 50 mg of calcium fructoborate; 100 to 500 mg of Terminalia chebula extract; and 100 to 500 mg of palm fruit extract; or consists substantially of about 4 mg of astaxanthin; about 95 mg of seaweed calcium composition, comprising 10 to 50 wt % of calcium; about 40 mg of undenatured collagen Type II; about 80 mg of hyaluronic acid; about 25 mg of calcium fructoborate; about 250 mg of Terminalia chebula extract; and about 350 mg of palm fruit extract. As used herein, “consist substantially of” refers to that the composition does not comprise other ingredients for managing pain, controlling inflammation, strengthening joints, or a combined function thereof, but it is not excluded that the composition might comprise ingredients for other purposes.

Astaxanthin. Astaxanthin is a natural lipid-soluble red pigment, which is a kind of keto-carotenoid having an IUPAC name, 3,3′-Dihydroxy-β,β-carotene-4,4′-dione. The structure of astaxanthin contains hydroxyl and ketone groups extending to a long carbon chain. The long carbon chain and the hydroxyl group provide astaxanthin with an amphiphilic nature, and the length of the long carbon chain suffice and enables the astaxanthin molecule across cell membranes; therefore, the astaxanthin molecule is able to exert anti-oxidation within and outside of a cell. Astaxanthin was first discovered and isolated from lobster shells, and then scientists subsequently identified the presence thereof in other organisms, such as Haematococcus pluvialis, Adonis aestivalis, Euphasia supurba, salmon, red snapper, etc. Among them, Haematococcus pluvialis is known to have the highest amount of astaxanthin. In some embodiments, the source of the astaxanthin of the present disclosure's composition is not limited; while in some specific embodiments, the astaxanthin is isolated or purified from Haematococcus pluvialis or Antarctic krill (Euphausia superba).

In some embodiments, the composition of the present disclosure comprises, in every 1000 mg, about 1, 2, 3, 4, 5, 6, 7, 8, 9, or 10 mg of astaxanthin, or any range defined by the foregoing two values, such as, 1 to 10 mg, 1 to 9 mg, 1 to 8 mg, 1 to 7 mg, 1 to 6 mg, 1 to 5 mg, 1 to 4 mg, 1 to 3 mg, 1 to 2 mg, 2 to 10 mg, 2 to 9 mg, 2 to 8 mg, 2 to 7 mg, 2 to 6 mg, 2 to 5 mg, 2 to 4 mg, 2 to 3 mg, 3 to 10 mg, 3 to 9 mg, 3 to 8 mg, 3 to 7 mg, 3 to 6 mg, 3 to 5 mg, 3 to 4 mg, 4 to 10 mg, 4 to 9 mg, 4 to 8 mg, 4 to 7 mg, 4 to 6 mg, 4 to 5 mg, 5 to 10 mg, 5 to 9 mg, 5 to 8 mg, 5 to 7 mg, 5 to 6 mg, 6 to 10 mg, 6 to 9 mg, 6 to 8 mg, 6 to 7 mg, 7 to 10 mg, 7 to 9 mg, 7 to 8 mg, 8 to 10 mg, 8 to 9 mg, or 9 to 10 mg of astaxanthin. In a specific embodiment, the composition of the present disclosure comprises, in every 1000 mg, about 4 mg of astaxanthin.

Depending on the optical activity of the hydroxyl groups at both ends of the astaxanthin structure, astaxanthin has left-handed (3S-3S′), racemic (3R-3S′), and right-handed (3R-3R′) structures. In some embodiments, the composition of the present disclosure comprises left-handed astaxanthin, racemic astaxanthin, and/or right-handed astaxanthin. In some embodiments, the astaxanthin of the present disclosure's composition comprises about 20 mol %, 25 mol %, 30 mol %, 35 mol %, 40 mol %, 45 mol %, 50 mol %, 55 mol %, 60 mol %, 65 mol %, 70 mol %, 75 mol %, 80 mol %, 85 mol %, 90 mol %, 95 mol %, or 100 mol % of left-handed astaxanthin, or any range defined by the foregoing two values, such as, 20 mol % to 100 mol %, 20 mol % to 95 mol %, 20 mol % to 90 mol %, 20 mol % to 85 mol %, 20 mol % to 80 mol %, 20 mol % to 75 mol %, 20 mol % to 70 mol %, 20 mol % to 65 mol %, 20 mol % to 60 mol %, 20 mol % to 55 mol %, 20 mol % to 50 mol %, 20 mol % to 45 mol %, 20 mol % to 40 mol %, 20 mol % to 35 mol %, 20 mol % to 30 mol %, 20 mol % to 25 mol %, 30 mol % to 100 mol %, 30 mol % to 95 mol %, 30 mol % to 90 mol %, 30 mol % to 85 mol %, 30 mol % to 80 mol %, 30 mol % to 75 mol %, 30 mol % to 70 mol %, 30 mol % to 65 mol %, 30 mol % to 60 mol %, 30 mol % to 55 mol %, 30 mol % to 50 mol %, 30 mol % to 45 mol %, 30 mol % to 40 mol %, 30 mol % to 35 mol %, 40 mol % to 100 mol %, 40 mol % to 95 mol %, 40 mol % to 90 mol %, 40 mol % to 85 mol %, 40 mol % to 80 mol %, 40 mol % to 75 mol %, 40 mol % to 70 mol %, 40 mol % to 65 mol %, 40 mol % to 60 mol %, 40 mol % to 55 mol %, 40 mol % to 50 mol %, 40 mol % to 45 mol %, 50 mol % to 100 mol %, 50 mol % to 95 mol %, 50 mol % to 90 mol %, 50 mol % to 85 mol %, 50 mol % to 80 mol %, 50 mol % to 75 mol %, 50 mol % to 70 mol %, 50 mol % to 65 mol %, 50 mol % to 60 mol %, 50 mol % to 55 mol %, 60 mol % to 100 mol %, 60 mol % to 95 mol %, 60 mol % to 90 mol %, 60 mol % to 85 mol %, 60 mol % to 80 mol %, 60 mol % to 75 mol %, 60 mol % to 70 mol %, 60 mol % to 65 mol %, 70 mol % to 100 mol %, 70 mol % to 95 mol %, 70 mol % to 90 mol %, 70 mol % to 85 mol %, 70 mol % to 80 mol %, 70 mol % to 75 mol %, 80 mol % to 100 mol %, 80 mol % to 95 mol %, 80 mol % to 90 mol %, 80 mol % to 85 mol %, 90 mol % to 100 mol %, 90 mol % to 95 mol %, or 95 mol % to 100 mol % of left-handed astaxanthin.

Seaweed calcium composition. The seaweed calcium composition of the present disclosure is a calcified seaweed composition, which comprises at least 10, 12, 15, 18, 20. 25, 30, 32, 35, 40, 45, or 50 wt % of calcium, or any range defined by the foregoing two values, such as, 10 to 50 wt %, 10 to 45 wt %, 10 to 40 wt %, 10 to 35 wt %, 10 to 32 wt %, 10 to 30 wt %, 10 to 25 wt %, 10 to 20 wt %, 10 to 18 wt %, 10 to 15 wt %, 10 to 12 wt %, 15 to 50 wt %, 15 to 45 wt %, 15 to 40 wt %, 15 to 35 wt %, 15 to 32 wt %, 15 to 30 wt %, 15 to 25 wt %, 15 to 20 wt %, 15 to 18 wt %, 20 to 50 wt %, 20 to 45 wt %, 20 to 40 wt %, 20 to 35 wt %, 20 to 32 wt %, 20 to 30 wt %, 20 to 25 wt %, 25 to 50 wt %, 25 to 45 wt %, 25 to 40 wt %, 25 to 35 wt %, 25 to 32 wt %, 25 to 30 wt %, 30 to 50 wt %, 30 to 45 wt %, 30 to 40 wt %, 30 to 35 wt %, 30 to 32 wt %, 32 to 50 wt %, 32 to 45 wt %, 32 to 40 wt %, or 32 to 35 wt % of calcium. In some specific embodiments, the seaweed is Lithothamnion sp.

In many embodiments, the seaweed calcium composition further comprises at least one micronutrition, which is selected from a group consisting of Magnesium, phosphorous, sulphur, iron, boron, fluoride, sodium, cobalt, copper, zinc, selenium, molybdenum, iodine, manganese, and nickel. In some specific embodiments, the seaweed calcium composition further comprises about 2, 2.2, 2.5, 3, 3.5, or 4 wt % of magnesium, or any range defined by the foregoing two values, such as 2 to 4 wt %, 2 to 3.5 wt %, 2 to 3 wt %, 2 to 2.5 wt %, 2.2 to 4 wt %, 2.2 to 3.5 wt %, 2.2 to 3 wt %, or 2.2 to 2.5 wt % of magnesium.

In some embodiments, the seaweed calcium composition comprises calcium (e.g., the calcified seaweed) of a honeycomb-like porous structure. Without wishing to be bound by any theories, the present disclosure contemplates that the honeycomb-like porous structure is favorable for providing an excellent bioavailability, and therefore, the seaweed calcium has a better bioavailability than that of calcium carbonate. In some embodiments, the seaweed calcium composition comprises calcium, which comprises a polymorph, and the polymorph comprises 60% to 70% of calcite, 20% to 30% of aragonite, and 10% to 20% of vaterite. In some specific embodiments, the polymorph comprises 65% calcite, 23% aragonite, and 12% vaterite.

In some embodiments, the composition of the present disclosure comprises, in every 1000 mg, about 50, 55, 60, 65, 70, 75, 80, 85, 90, 95, or 100 mg of seaweed calcium composition (wherein the seaweed calcium composition comprises at least 10 wt % of calcium), or any range defined by the foregoing two values, such as, 50 to 100 mg, 50 to 95 mg, 50 to 90 mg, 50 to 85 mg, 50 to 80 mg, 50 to 75 mg, 50 to 70 mg, 50 to 60 mg, 50 to 55 mg, 60 to 100 mg, 60 to 95 mg, 60 to 90 mg, 60 to 85 mg, 60 to 80 mg, 60 to 75 mg, 60 to 70 mg, 70 to 100 mg, 70 to 95 mg, 70 to 90 mg, 70 to 85 mg, 70 to 80 mg, 70 to 75 mg, 80 to 100 mg, 80 to 95 mg, 80 to 90 mg, 80 to 85 mg, 90 to 100 mg, 90 to 95 mg, or 50 to 100 mg of seaweed calcium composition, wherein the seaweed calcium composition comprises at least 10 wt % or other amounts of calcium, as described herein. In a specific embodiment, the composition of the present disclosure comprises, in every 1000 mg, about 95% of the seaweed calcium composition.

Collagen. In some embodiments, the collagen of the composition of the present disclosure is Collagen Type II. In many specific embodiments, the collagen is undenatured Collagen Type II, which has a triple helix structure. In many specific embodiments, the collagen is not a hydrolyzed collagen. Without wishing to be bound by any theories, the present disclosure contemplates that hydrolyzed collagen has a smaller molecule and is easier for humans to uptake and break down into the materials needed to synthesize collagen in the body; undenatured Collagen Type II's mechanism of action with the human body is to regulate humoral immune response and cellular immune response via established oral tolerance by interacting with gut-associated lymphoid tissues. It is believed that undenatured collagen Type II can be absorbed by Treg cells and, therefore, activate the Treg cells in the Peyer's patch of the human small intestine. The activated Treg cells, while circulating in the human body, can identify collagen type II at an inflammation site at a joint area and release regulatory factors to alleviate inflammation.

In some embodiments, the composition of the present disclosure comprises, in every 1000 mg, about 20, 25, 30, 35, 40, 45, 50, 55, or 60 mg of collagen, or any range defined by the foregoing two values, such as 20 to 60 mg, 20 to 55 mg, 20 to 50 mg, 20 to 45 mg, 20 to 40 mg, 20 to 35 mg, 20 to 30 mg, 20 to 25 mg, 30 to 60 mg, 30 to 55 mg, 30 to 50 mg, 30 to 45 mg, 30 to 40 mg, 30 to 35 mg, 35 to 60 mg, 35 to 55 mg, 35 to 50 mg, 35 to 45 mg, or 35 to 40 mg of collagen. In a specific embodiment, the composition of the present disclosure comprises, in every 1000 mg, about 40 mg of collagen.

Hyaluronic acid. Hyaluronic acid is a disaccharide polymer structure composed of D-glucuronic acid and N-acetylglucosamine. Without wishing to be bound by any theories, the present disclosure contemplates that hyaluronic acid, after binding the TLR-4 receptor on epithelial cells in the intestine, can promote the secretion of the anti-inflammatory factor IL-10, thereby increasing the activity of Suppressor of Cytokine Signaling 3 (SCOCS3) and therefore regulating inflammation. Moreover, without wishing to be bound by any theories, the present disclosure contemplates that hyaluronic acid's functionality can be closely related to its molecular weight. In some embodiments, the hyaluronic acid of the present disclosure's composition has a weight-average molecule weight of 5, 10, 20, 50, 100, 200, 500, 600, 700, 800, 900, or 1000 kDa, or any range defined by the foregoing two values, such as 5 to 1000 kDa, 5 to 900 kDa, 5 to 800 kDa, 5 to 700 kDa, 5 to 600 kDa, 5 to 500 kDa, 5 to 200 kDa, 5 to 100 kDa, 5 to 50 kDa, 5 to 20 kDa, 20 to 1000 kDa, 20 to 900 kDa, 20 to 800 kDa, 20 to 700 kDa, 20 to 600 kDa, 20 to 500 kDa, 20 to 200 kDa, 20 to 100, 20 to 50 kDa, 200 to 1000 kDa, 200 to 900 kDa, 200 to 800 kDa, 200 to 700 kDa, 200 to 600 kDa, 200 to 500 kDa, 500 to 1000 kDa, 500 to 900 kDa, 500 to 800 kDa, 500 to 700 kDa, or 500 to 600 kDa.

In some embodiments, the present disclosure's composition comprises, in every 1000 mg, about 50, 55, 60, 65, 70, 75, 80, 85, 90, 95, or 100 mg of hyaluronic acid, or any range defined by the foregoing two values, such as, 50 to 100 mg, 50 to 90 mg, 50 to 80 mg, 50 to 70 mg, 50 to 60 mg, 60 to 100 mg, 60 to 90 mg, 60 to 80 mg, 60 to 75 mg, 60 to 70 mg, 70 to 100 mg, 70 to 90 mg, 70 to 80 mg, 80 to 100 mg, 80 to 90 mg, or 90 to 100 mg. In some embodiments, the present disclosure's composition comprises, in every 1000 mg, about 80 mg of hyaluronic acid. In some embodiments, the hyaluronic acid is isolated from animals (e.g., chicken comb) or microorganisms.

Calcium fructoborate. Calcium fructoborate is a mineral borate complex, comprising a chemical formula of Ca [C₆H₁₀O₆)₂B]₂. In some embodiments, the present disclosure's composition comprises, in every 1000 mg, 10, 15, 20, 25, or 30 mg of calcium fructoborate, or any range defined by the foregoing two values, such as 10 to 30 mg, 10 to 25 mg, 10 to 20 mg, 10 to 15 mg, 15 to 30 mg, 15 to 25 mg, 15 to 20 mg, or 20 to 30 mg of calcium fructoborate. In some specific embodiments, the present disclosure's composition comprises, in every 1000 mg, about 25 mg of calcium fructoborate.

Terminalia chebula extract. In some embodiments, the Terminalia chebula extract of the present disclosure's composition is a water-soluble extract, for example, an extract extracted from a peel of a Terminalia chebula fruit. In some embodiments, the Terminalia chebula extract comprises ellagitannin, chebulinic acid, or a combination thereof. In some embodiments, the Terminalia chebula extract further comprises hydrolyzable tannin (hydrolyzable tannoid).

In some specific embodiments, the Terminalia chebula extract comprises 10, 12, 15, 18, 20, 25, or 30 wt % of ellagitannin, or any range defined by the foregoing two values, such as 10 to 30 wt %, 10 to 25 wt %, 10 to 20 wt %, 10 to 15 wt %, 15 to 30 wt %, 15 to 25 wt %, 15 to 20 wt %, 15 to 18 wt %, 20 to 30 wt %, or 20 to 25 wt % of ellagitannin. In many specific embodiments, the Terminalia chebula extract comprises about 5, 6, 7, 8, 9, 10, 12, 15, 18, 20, or 25 wt % of chebulinic acid, or any range defined by the foregoing two values, such as, 5 to 25 wt %, 5 to 20 wt %, 5 to 15 wt %, 5 to 10 wt %, 8 to 25 wt %, 8 to 20 wt %, 8 to 15 wt %, 8 to 10 wt %, 15 to 25 wt %, or 15 to 20 wt % of chebulinic acid. In some specific embodiments, the Terminalia chebula extract comprises about 5, 10, 15, 20, 25, 30, 35, 40, 45, or 50 wt % of hydrolyzable tannin, or any range defined by the foregoing two values, such as 5 to 50 wt %, 5 to 45 wt %, 5 to 40 wt %, 5 to 35 wt %, 5 to 30 wt %, 5 to 25 wt %, 5 to 20 wt %, 5 to 15 wt %, 5 to 10 wt %, 15 to 50 wt %, 15 to 45 wt %, 15 to 40 wt %, 15 to 35 wt %, 15 to 30 wt %, 15 to 25 wt %, 15 to 20 wt %, 25 to 50 wt %, 25 to 45 wt %, 25 to 40 wt %, 25 to 35 wt %, 25 to 30 wt %, 35 to 50 wt %, 35 to 45 wt %, or 35 to 40 wt % hydrolysable tannin. In a specific embodiment, the Terminalia chebula extract comprises 15 to 30 wt % of ellagitannin, 8 to 25 wt % of chebulinic acid, and 5 to 45 wt % of hydrolyzable tannin.

In some embodiments, the Terminalia chebula extract was obtained by a method comprising: obtaining a peel of a Terminalia chebula fruit, grinding the peel to obtain a powder, extracting the powder using water to obtain a Terminalia chebula peel water-soluble extract, and drying the Terminalia chebula peel water-soluble extract to obtain a powder-form extract. In many embodiments, the drying is conducted by spray drying or freeze drying. In some embodiments, the extracting is conducted at 25 degrees Celsius for 10 to 20 hours (e.g., 12 to 16 hours, or 12, 13, 14, 15, or 16 hours) or at 40 degrees Celsius for 1 to 10 hours (e.g., 1 to 6 hours, or 1, 2, 3, 4, 5, or 6 hours).

In some embodiments, the present disclosure's composition comprises, in every 1000 mg, about 200, 220, 240, 250, 260, 280, or 300 mg of the Terminalia chebula extract, or any range defined by the foregoing two values, such as 200 to 300 mg, 200 to 280 mg, 200 to 260 mg, 200 to 250 mg, 200 to 240 mg, 200 to 220 mg, 220 to 300 mg, 220 to 280 mg, 220 to 260 mg, 220 to 250 mg, or 220 to 240 mg of the Terminalia chebula extract. In some specific embodiments, the present disclosure's composition comprises, in every 1000 mg, about 250 mg of the Terminalia chebula extract.

Palm fruit extract. In some embodiments, the palm fruit extract of the present disclosure's composition comprises palmitoylethanolamide (PEA). In contrast to other inflammatory mediators, the amount of PEA in human tissue will decrease during inflammation. Without wishing to be bound by any theories, the present disclosure contemplates that increasing the PEA amount can increase the concentration of anandamide (AEA) in the human body and, therefore, alleviate pain. Besides, PEA is capable of activating the CB2 receptor, thereby controlling inflammation via the COX-2 pathway.

In many embodiments, the palm fruit extract is obtained by obtaining a palm fruit; threshing the palm fruit to obtain a threshed palm fruit; boiling the threshed palm fruit; drying the boiled palm fruit and grinding the dried palm fruit to obtain a powder, and extracting the powder using ethanol to obtain a palm fruit crude extract. In some embodiments, the method comprises concentrating the palm fruit crude extract to obtain a first extract. In some embodiments, the method further comprises extracting the first extract using ethanol and drying the product to obtain a second extract. In some embodiments, the method further comprises extracting the second extract using ethanol and drying the product to obtain a third extract. In some embodiments, the method further comprises drying the first extract, the second extract, or the third extract to obtain a powder of the palm fruit extract. In many embodiments, the drying is conducted by vacuum drying or freeze drying. In some embodiments, the method further comprises mixing the palm fruit extract powder with an excipient, wherein the excipient can be, but is not limited to, lecithin, coconut oil, or silicon dioxide.

In some embodiments, the present disclosure's composition comprises, in every 1000 mg, about 200, 225, 250, 275, 300, 325, 350, 375, or 400 mg of the palm fruit extract, or any range defined by the foregoing two values, such as 200 to 400 mg, 200 to 375 mg, 200 to 350 mg, 200 to 300 mg, 200 to 250 mg, 250 to 400 mg, 250 to 375 mg, 250 to 350 mg, 250 to 300 mg, 300 to 400 mg, 300 to 375 mg, or 300 to 350 mg of the palm fruit extract. In some specific embodiments, the present disclosure's composition comprises, in every 1000 mg, about 350 mg of the palm fruit extract.

The preparation of the present disclosure's composition. The present disclosure's composition can be prepared by more than one method as long as the prepared composition comprises the ingredients at the desired amount as disclosed in the present disclosure. In some specific embodiments, the present disclosure's composition is prepared by a method comprising obtaining each individual ingredient of required amounts, for example, weighing and obtaining 100 to 200 grams of Haematococcus pluvialis powder (containing 2.5% of astaxanthin), weighing and obtaining 50 to 120 grams of red algae calcium, weighing and obtaining 20 to 60 grams of undenatured type II collagen, weighing and obtaining about 80 grams of chicken comb extract (containing 60 to 75% hyaluronic acid), weighing and obtaining 10 to 40 grams of calcium fructoborate, weighing and obtaining 100 to 300 grams of Terminalia chebula extract, and weighing and obtaining 200 to 500 grams of palm fruit extract; and mixing the ingredients evenly to obtain a 1000-grams of raw material composition. The raw material composition can then be formulated into a desired formulation as consumer products.

In some embodiments, the composition can be formulated as an oral composition. In many embodiments, when the composition is an oral composition, it can be in the form of powder, granules, tablets, capsules, solutions, suspensions, or combinations thereof.

In some embodiments, the composition can further comprise a pharmaceutically acceptable carrier, including but not limited to a binder, diluent, disintegrant, filler, glidant, lubricant, coloring agent, preservative, sweetener, surfactant, solvent, coating agent, or a combination thereof.

### Treatment Methods

Another aspect of the present disclosure provides a method for treating arthritis. In some embodiments, the arthritis is degenerative arthritis (also known as Osteoarthritis). The method of the present disclosure comprises administering the composition for treating arthritis of the present disclosure to a subject. In many specific embodiments, the composition for treating arthritis is as described herein.

In many embodiments, the subject is diagnosed or is self-diagnosed to have arthritis or symptoms thereof. In some specific embodiments, the subject is a mammal, for example, but not limited to humans, dogs, cats, horses, cattle, monkeys, rabbits, mice, or sheep.

In some embodiments, the subject is administered with the composition at an effective amount. In many specific embodiments, the effective amount is 200, 250, 300, 350, 400, 450, 500, 550, 600, 650, 700, 750, 800, 850, 900, 950, 1000, 1200, 1500, 1750, or 2000 mg per 60 Kg-Body Weight, or any range defined by the foregoing two values, such as 200 to 2000 mg, 200 to 1750 mg, 200 to 1500 mg, 200 to 1200 mg, 200 to 1000 mg, 200 to 750 mg, 200 to 500 mg, 200 to 300 mg, 300 to 2000 mg, 300 to 1750 mg, 300 to 1500 mg, 300 to 1200 mg, 300 to 1000 mg, 300 to 750 mg, 300 to 500 mg, 500 to 2000 mg, 500 to 1750 mg, 500 to 1500 mg, 500 to 1200 mg, 500 to 1000 mg, 500 to 750 mg, 800 to 2000 mg, 800 to 1750 mg, 800 to 1500 mg, 800 to 1200 mg, 800 to 1000 mg, 1000 to 2000 mg, 1000 to 1750 mg, 1000 to 1500 mg, 1000 to 1200 mg, 1500 to 2000 mg, 1500 to 1750 mg, 1500 to 1500 mg, or 1500 to 1200 mg. In some embodiments, the effective amount is a daily amount administered to the subject.

In some embodiments, the administration is conducted via oral route. In many embodiments, the administration is conducted 1, 2, 3, 4, 5, 6, 7, 8, 9, or 10 times, or more, or any range defined by the foregoing two values, such as 1 to 10 times, 1 to 8 times, 1 to 6 times, 1 to 4 times, 3 to 10 times, 3 to 8 times, 3 to 6 times, or 3 to 4 times. In some embodiments, when the administration is conducted at least 2 times, the interval of the two administrations is about 1 hour, 4 hours, 8 hours, 12 hours, 16 hours, 24 hours, 2 days, 4 days, 1 week, 2 weeks, 1 month, 3 months, or 6 months, or any range defined by the foregoing two values, such as 1 to 4 hours, 1 to 8 hours, 1 to 24 hours, 24 hours to 2 days, 2 to 4 days, 1 week to 1 month, 1 to 3 months, 1 to 6 months, or 3 to 6 months. In certain embodiments when the administration is conducted at least 3 times, the intervals between each consecutive administration can be the same or different. For example, the interval between the first and second administration is 4 hours, while the interval between the second and third administration is 8 hours.

### Definitions

As described herein, the term “comprising,” “including,” or “having” refers to the presence of the specified features, values, steps, operations, members, components, and/or combinations thereof but does not exclude the possibility of the presence of other features, values, steps, operations, members, components, and/or combinations thereof. As described herein, only the phrase “consisting of” may be interpreted as a closed-ended transition, and only the term “consisting essentially of” may be interpreted as a semi-closed transition.

As described herein, an “effective amount” of a composition refers to an amount that can achieve the described clinical outcome or therapeutic effect within a specific desired timeframe. An effective amount may be achieved through multiple administrations. If the subject receiving the administration or treatment with the composition has been confirmed to have the described disease or symptom, the effective amount may be referred to as a “therapeutically effective amount.” For a subject at risk of developing the disease or symptom (e.g., recurrence), the effective amount administered to the individual may be referred to as a “prophylactically effective amount.”

As described herein, the term “administration” refers to the act of providing the composition to a subject, where the subject may self-administer or be administered the composition. The term “administration” used in the present disclosure is not limited to a specific route, quantity, frequency, or method through which the subject receives the composition.

### Example 1: The Therapeutic Effect of the Present Disclosure's Composition in Treating Arthritis

This experiment tested the efficacy of an exemplary composition according to the present disclosure in treating arthritis by using a rat model. The Sprague-Dawley rats used in this experiment were purchased from BioLASCO Taiwan Co. Ltd. and were maintained according to the supplier's instructions before the experiments. To establish an animal model of degenerative arthritis, the cruciate ligament and medial meniscus of the right knee of the rat were removed, causing instability in the movement of the right knee joint, whereby the rats were induced to develop degenerative arthritis.

Next, the exemplary composition according to the present disclosure was prepared. The composition comprises, in every 1000 mg, about 160 mg of astaxanthin composition (which comprises 2.5% of astaxanthin), 95 mg of seaweed calcium composition (which comprises about 32 wt % of calcium), 40 mg of undenatured collagen Type II, 80 mg of hyaluronic acid, 25 mg of calcium fructoborate, 250 mg of Terminalia chebula extract, and 350 mg of palm fruit extract. The effective amount of the exemplary composition for humans was 1000 mg/60 Kg-Body Weight. Based on that effective amount, a suitable dosage for the experimental rats was calculated as about 105.4 mg/Kg BW. Each rat of the experiment group (which had 18 rats) was administered with the exemplary composition once per week orally, and each rat of the control group (which also had 18 rats) was administered with RO water of the same amount.

### Observation

After being administered with the exemplary composition or water, the rats were maintained according to the supplier's instructions. Their bodyweight, water intake, and food intake were recorded to monitor their physiological condition. Except that, the width of the rat's right knee joint (where the surgery was performed) was measured and record once per week, which included initial width before the surgery, and the ratio of width of the right knee and the left knee was calculated accordingly. Then, 3 rats from each group were sacrificed at the first month after the surgery, another 3 from each group at the second month, and the rest 12 rats from each group were sacrificed at the third month after the surgery to observe their knee joint using Hematoxylin & Eosin (H&E). The steps for the H&E observation comprised dissecting the rat's knee joint and fixing it in 10% neutral formalin solution for 24 hours, decalcifying the fixed knee joint using a Leica® Decalcifier reagent, trimming the decalcified knee joint, and preparing it into paraffin tissue sections. The obtained tissue sections were then stained using H&E to evaluate the width of the cartilage matrix loss, cartilage degeneration score, total tibial cartilage degeneration width, significant tibial cartilage degeneration width, and zonal depth ratio of lesions.

Width of cartilage matrix loss. The loss of cartilage matrix mostly happens when the surface of the cartilage is damaged into fibrous structures with matrix loss. The damage can then worsen into a deeper and broader extent of matrix loss and eventually result in complete loss of the matrix. This assessment included calculating the extent of cartilage thickness (width) loss. Based on the severity of matrix loss, the cartilage matrix loss is classified into: superficial layer width (0% depth zone, representing any extent of matrix loss within the intact cartilage area spanning both sides), middle layer width (50% depth zone, where the cartilage matrix loss has reached the middle layer), and tidemark layer width (100% depth zone, where the matrix loss has severely progressed to the tidemark). The three widths were summed up to present the extent of the cartilage matrix loss in micrometers.

Cartilage degeneration score. This assessment was primarily based on the damage and death of cartilage cells. To that end, the medial weight-bearing platform of the tibia in the knee joint was divided into three equal zones: the outer zone (starting from the inner edge of the joint), the middle zone, and the inner zone (adjacent to the cruciate ligament at the center of the knee joint). The severity of damage is graded on a 0-5 scale for each zone. Each zone has a maximum score of 5, with 0 indicating no damage and 5 indicating severe damage exceeding 75%. The total score for all three zones is 15 points.

Total tibial cartilage degeneration width. This assessment determined the width of any degenerative features observed on the surface of the tibial cartilage, presented in micrometers.

Significant tibial cartilage degeneration width. This assessment determined the width of any significant degenerative features (the total thickness from the cartilage surface to tidemark where over 50% of the cartilage cells are damaged or dead) of the tibial cartilage, presented in micrometers.

Zonal depth ratio of lesions. This assessment determined the ratio of the depth of the degenerative cartilage. The “cartilage degeneration score” was divided into three zones, and the ratio of lesion depth to the total cartilage thickness (from the cartilage surface to the tidemark) in each zone was calculated as a score. A score of 0 indicates no cartilage wear, a score of 1 indicates full-thickness wear and the maximum total score is 3.

Statistical Analysis: All values are expressed as mean±SEM in figures and mean±SD in tables. Statistical analyses were performed using the GraphPad Prism 6 software for one-way analysis of variance (ANOVA), and differences between treatments were analyzed using Student's t-test. A p-value of <0.05 was considered statistically significant

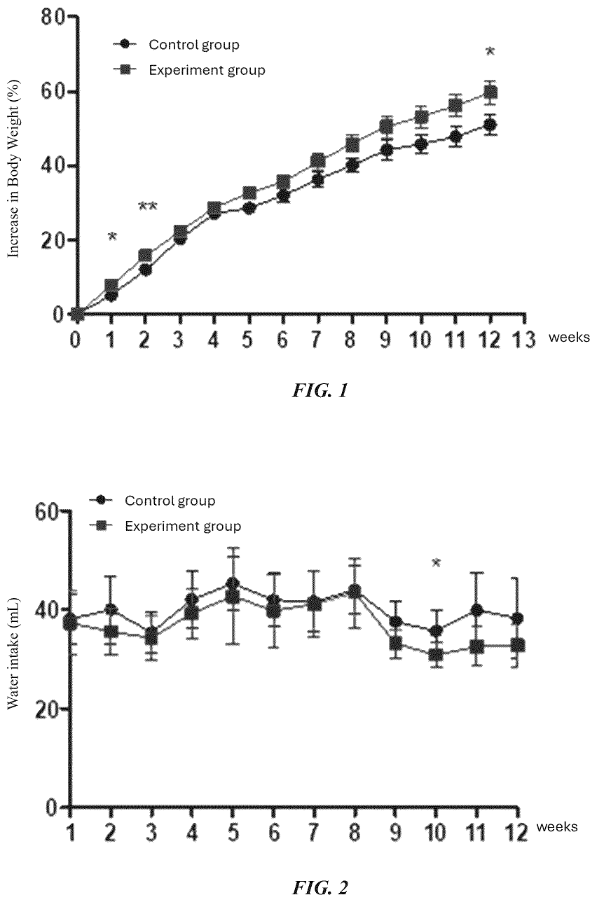

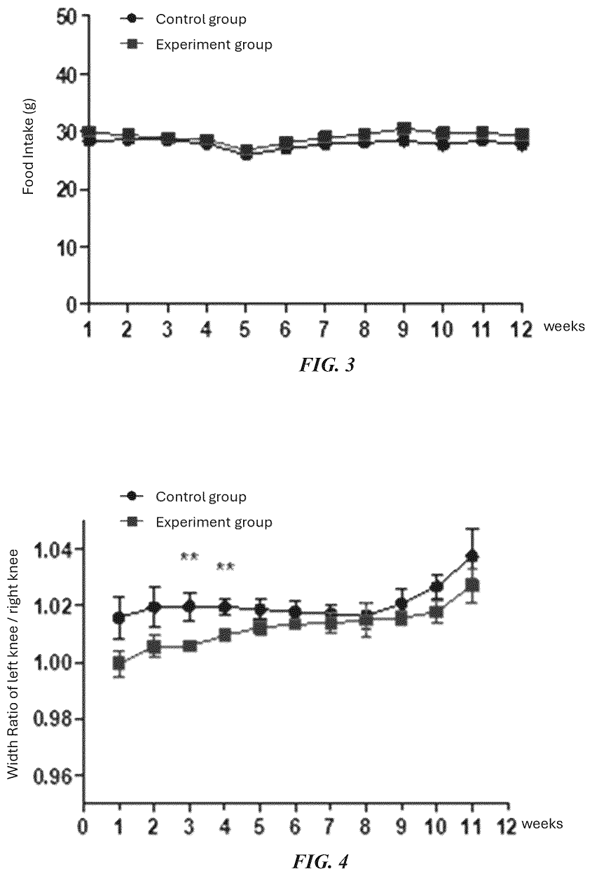

Results. First of all, the body weight of the rats in the experiment group was stably heavier than that of the rats in the control group (FIG. 1), and both groups did not have significant changes during the experiments. Besides, the water intake (FIG. 2) and food intake (FIG. 3) of the rats were also maintained stably during the period. These records showed that the rats were maintained properly, and the exemplary composition did not significantly affect the normal physiology of the rats. In addition, the measurements of the width of the knee joint showed that, during the experiments, the rats of the experiment group had a lower knee joint width ratio than that of the control group. The difference in the width ratio of the knee joint is statistically significant, especially in Week 3 and Week 4 (FIG. 4).

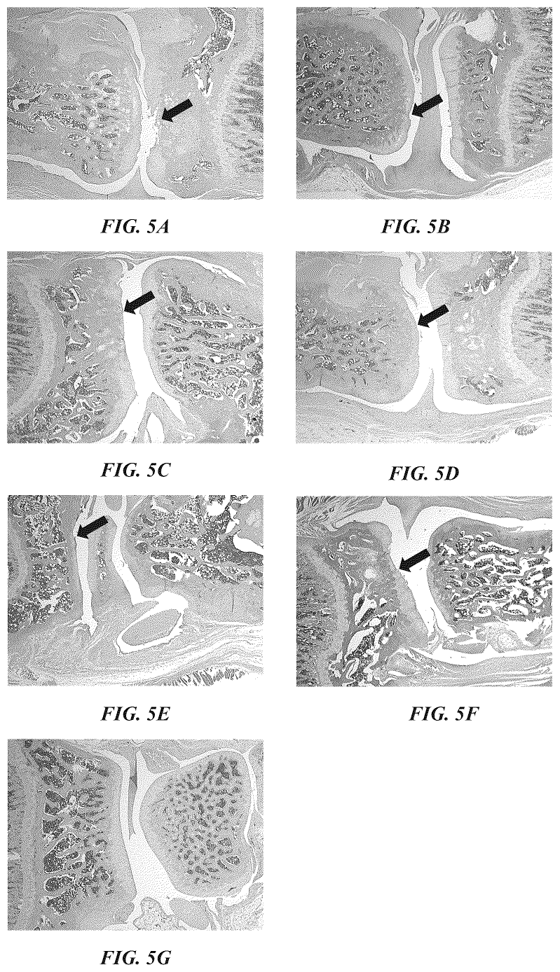

H&E staining and scoring are shown in Table 1, Table 2, Table 3, and Table 4; the H&E photos are shown in FIG. 5A, FIG. 5B, FIG. 5C, FIG. 5D, FIG. 5E, FIG. 5F, and FIG. 5G. At the tibial end of the joint, the arthritis OA score for the control group was 13.4±8.64; while that of the experiment group was 8.0±7.58. The arthritis OA score for the control group one month after surgery was 5.3±1.15, while the arthritis OA score for the experiment group was 5.0±1.00. Two months after surgery, the arthritis OA score for the control group was 12.0±6.92, and that of the experiment group was 13.3±9.45. These results showed that the control group and the experiment group were not significantly different at the tibial end of the joint.

As to the femur end of the joint, the arthritis OA score for the control group was 13.8±5.99, while the experiment group scored 7.5±4.30. The arthritis OA score for the control group one month after surgery was 5.0±6.08, and the experiment group had improved to 2.0±2.00. Two months after surgery, the arthritis OA score for the control group was 6.6±5.03, and that of the experiment group was 2.0±2.00. These results showed that the experiment group had significant improvement in the severity of knee joint lesions at the femur end compared with the control group.

In terms of lesion scoring on the joint surfaces at both ends of the knee joint, The OA score for the joint surfaces at both ends of the knee in the control group was 13.6±7.04, while the OA score for the joint surfaces at both ends in the experimental group was 7.7±4.66. One month after the surgery, the control group scored 5.1±3.40, and the experiment group scored 3.5±1.50. The mean median lesion severity score for the control group two months after surgery was 9.3±5.85, while the OA score of the experimental group at that time was 7.6±5.03. The results showed that, in terms of OA scores for the joint surfaces at both ends, the experimental group exhibited significant improvement in the severity of knee joint lesions compared to the control group.

TABLE 1
Severity of knee joint lesions of the control group. C101, C102,
and C201 are the scores one month after surgery; C202, C301,
and C302 are the scores two months after surgery (OA score
was calculated by multiplying the extent with the stage; the
average of the joint surfaces at both ends is the average of
the summed OA scores of the tibial and femoral ends).

| No. | C101 | C102 | C201 | C202 | C301 | C302 |
|---|---|---|---|---|---|---|
| Tibial end of the joint | | | | | | |
| Extent (lesion severity) | 2 | 6 | 3 | 5 | 4 | 4 |
| Stage (lesion area) | 2 | 1 | 2 | 4 | 2 | 2 |
| OA score | 4 | 6 | 6 | 20 | 8 | 8 |
| Femoral end of the joint | | | | | | |
| Extent (lesion severity) | 1 | 2 | 4 | 6 | 3 | 2 |
| Stage (lesion area) | 1 | 1 | 3 | 2 | 2 | 1 |
| OA score | 1 | 2 | 12 | 12 | 6 | 2 |
| Average at both ends | 2.5 | 4 | 9 | 16 | 7 | 5 |

TABLE 2
Severity of knee joint lesions of the experiment group. T101,
T102, and T201 are the scores one month after surgery; T202,
T301, and T302 are the scores two months after surgery (OA
score was calculated by multiplying the extent with the stage;
the average of the joint surfaces at both ends is the average
of the summed OA scores of the tibial and femoral ends).

| No. | T101 | T102 | T201 | T202 | T301 | T302 |
|---|---|---|---|---|---|---|
| Tibial end of the joint | | | | | | |
| Extent (lesion severity) | 4 | 2 | 5 | 6 | 2 | 5 |
| Stage (lesion area) | 1 | 3 | 1 | 4 | 3 | 2 |
| OA score | 4 | 6 | 5 | 24 | 6 | 10 |
| Femoral end of the joint | | | | | | |
| Extent (lesion severity) | 0 | 2 | 2 | 2 | 0 | 4 |
| Stage (lesion area) | 0 | 2 | 1 | 1 | 0 | 1 |
| OA score | 0 | 4 | 2 | 2 | 0 | 4 |
| Average at both ends | 2 | 5 | 3.5 | 13 | 3 | 7 |

TABLE 3
Severity of knee joint lesions of the control group three months after surgery. (The OA score
was calculated by multiplying the extent by the stage; the average of the joint surfaces
at both ends is the average of the summed OA scores of the tibial and femoral ends).

| No. | C401 | C501 | C502 | C601 | C602 | C701 | C702 | C801 | C802 | C901 |
|---|---|---|---|---|---|---|---|---|---|---|
| Tibial end of the joint | | | | | | | | | | |
| Extent (lesion severity) | 2 | 3 | 5 | 6 | 3 | 4 | 6 | 6 | 3 | 6 |
| Stage (lesion area) | 2 | 1 | 3 | 2 | 1 | 4 | 4 | 4 | 3 | 4 |
| OA score | 4 | 3 | 15 | 12 | 3 | 16 | 24 | 24 | 9 | 24 |
| Femoral end of the joint | | | | | | | | | | |
| Extent (lesion severity) | 5 | 2 | 5 | 4 | 5 | 5 | 5 | 5 | 3 | 5 |
| Stage (lesion area) | 2 | 3 | 2 | 4 | 2 | 4 | 4 | 4 | 2 | 4 |
| OA score | 10 | 6 | 10 | 16 | 10 | 20 | 20 | 20 | 6 | 20 |
| Average at both ends | 7 | 4.5 | 12.5 | 14 | 6.5 | 18 | 22 | 22 | 7.5 | 22 |

TABLE 4
Severity of knee joint lesions of the experiment group three months after surgery. (The OA
score was calculated by multiplying the extent by the stage; the average of the joint surfaces
at both ends is the average of the summed OA scores of the tibial and femoral ends).

| No. | T402 | T501 | T502 | T601 | T602 | T701 | T801 | T802 | T901 | T902 |
|---|---|---|---|---|---|---|---|---|---|---|
| Tibial end of the joint | | | | | | | | | | |
| Extent (lesion severity) | 2 | 6 | 2 | 3 | 2 | 6 | 3 | 4 | 6 | 6 |
| Stage (lesion area) | 1 | 1 | 1 | 1 | 2 | 4 | 1 | 3 | 1 | 3 |
| OA score | 2 | 6 | 2 | 3 | 4 | 24 | 3 | 12 | 6 | 18 |
| Femoral end of the joint | | | | | | | | | | |
| Extent (lesion severity) | 5 | 2 | 0 | 5 | 4 | 2 | 2 | 4 | 3 | 4 |
| Stage (lesion area) | 2 | 2 | 0 | 3 | 2 | 4 | 2 | 3 | 2 | 2 |
| OA score | 10 | 4 | 0 | 15 | 8 | 8 | 4 | 12 | 6 | 8 |
| Average at both ends | 6 | 5 | 1 | 9 | 6 | 16 | 3.5 | 12 | 6 | 13 |

The rat model experiments exhibited that the composition of the present disclosure can alleviate the severity and the area of arthritis, especially at the femoral end of the joint. Besides, significant differences in the recovery from arthritis can be observed after four times of administration. Accordingly, the present disclosure's composition's efficacy in alleviating arthritis symptoms and facilitating recovery was evident.

### EMBODIMENTS

Embodiment 1: A composition for treating arthritis, comprising, in every 1000 mg, 1 to 10 mg of astaxanthin; 30 to 200 mg of seaweed calcium composition, comprising at least 10 wt % of calcium; 5 to 100 mg of collagen; 20 to 150 mg of hyaluronic acid; 5 to 50 mg of calcium fructoborate; 100 to 500 mg of Terminalia chebula extract; and 100 to 500 mg of palm fruit extract.

Embodiment 2: The composition of Embodiment 1, comprising, in every 1000 mg, 3 to 8 mg of astaxanthin.

Embodiment 3: The composition of Embodiment 1 or Embodiment 2, wherein the astaxanthin comprises 20% to 100% of left-handed astaxanthin.

Embodiment 4: The composition of any one of Embodiments 1 to 3, comprising, in every 1000 mg, 50 to 100 mg of seaweed calcium composition.

Embodiment 5: The composition of any one of Embodiments 1 to 4, wherein the seaweed calcium composition comprises 10 to 50 wt % of calcium.

Embodiment 6: The composition of any one of Embodiments 1 to 5, wherein the seaweed calcium composition further comprises at least one micronutrition, which is selected from the group consisting of Magnesium, phosphorous, sulphur, iron, boron, fluoride, sodium, cobalt, copper, zinc, selenium, molybdenum, iodine, manganese, and nickel.

Embodiment 7: The composition of any one of Embodiments 1 to 6, wherein the seaweed calcium composition comprises a honeycomb-like porous structure.

Embodiment 8: The composition of any one of Embodiments 1 to 7, wherein the calcium of the seaweed calcium composition comprises is of a polymorph, comprising 60% to 70% of calcite, 20% to 30% of aragonite, and 10% to 20% of vaterite.

Embodiment 9: The composition of any one of Embodiments 1 to 8, comprising, in every 1000 mg, 20 to 60 mg of collagen.

Embodiment 10: The composition of any one of Embodiments 1 to 9, wherein the collagen is Collagen Type II.

Embodiment 11: The composition of Embodiment 10, wherein the collagen is undenatured Collagen Type II.

Embodiment 12: The composition of any one of Embodiments 1 to 11, comprising, in every 1000 mg, 50 to 100 mg of hyaluronic acid.

Embodiment 13: The composition of any one of Embodiments 1 to 12, comprising, in every 1000 mg, 10 to 30 mg of calcium fructoborate.

Embodiment 14: The composition of any one of Embodiments 1 to 13, comprising, in every 1000 mg, 200 to 300 mg of Terminalia chebula extract.

Embodiment 15: The composition of any one of Embodiments 1 to 14, wherein the Terminalia chebula extract comprises ellagitannin, chebulinic acid, hydrolyzable tannin, or a combination thereof.

Embodiment 16: The composition of any one of Embodiments 1 to 15, wherein the Terminalia chebula extract is obtained by a method, comprising: obtaining a peel of a Terminalia chebula fruit, grinding the peel to obtain a powder, extracting the powder using water to obtain a Terminalia chebula peel water-soluble extract, and drying the Terminalia chebula peel water-soluble extract to obtain a powder-form extract.

Embodiment 17: The composition of any one of Embodiments 1 to 16, comprising, in every 1000 mg, 200 to 400 mg of palm fruit extract.

Embodiment 18: The composition of any one of Embodiments 1 to 17, wherein the palm fruit extract comprises palmitoylethanolamide.

Embodiment 19: The composition of any one of Embodiments 1 to 18, comprising, in every 1000 mg, about 4 mg of astaxanthin; about 95 mg of seaweed calcium composition, comprising 10 to 50 wt % of calcium; about 40 mg of undenatured collagen Type II; about 80 mg of hyaluronic acid; about 25 mg of calcium fructoborate; about 250 mg of Terminalia chebula extract; and about 350 mg of palm fruit extract.

Embodiment 20: The composition of any one of Embodiments 1 to 19, wherein the arthritis is degenerative arthritis.

Embodiment 21: The composition of any one of Embodiments 1 to 20, wherein the composition is an oral composition.

Embodiment 22: The composition of any one of Embodiments 1 to 21, further comprising a pharmaceutically acceptable carrier.

Embodiment 23: A method for treating arthritis, comprising administering the composition of any one of Embodiments 1 to 22 to a subject.

Embodiment 24: The method of Embodiment 23, wherein the composition has an effective amount of 200 to 2000 mg/per 60 Kg-Body Weight.

Embodiment 25: The method of Embodiment 24, wherein the composition has an effective amount of 500 to 2000 mg/per 60 Kg-Body Weight.

Embodiment 26: The method of Embodiment 25, wherein the composition has an effective amount of 1000 mg/per 60 Kg-Body Weight.

Embodiment 27: The method of any one of Embodiments 23 to 26, wherein the administering is conducted via oral route.

Embodiment 28: The method of any one of Embodiments 23 to 27, wherein the subject is administered with the composition at least two times.

Embodiment 29: A use of a mixture for preparation of a composition for treating arthritis, wherein the mixture comprises astaxanthin, seaweed calcium composition, collagen, hyaluronic acid, calcium fructoborate, Terminalia chebula extract, and palm fruit extract, and wherein the composition for treating arthritis is according to the composition of any one of Embodiments 1 to 22.

What is claimed is:

1. A composition for treating arthritis, comprising, in every 1000 mg,
1 to 10 mg of astaxanthin;
30 to 200 mg of seaweed calcium composition, comprising at least 10 wt % of calcium;
5 to 100 mg of collagen;
20 to 150 mg of hyaluronic acid;
5 to 50 mg of calcium fructoborate;
100 to 500 mg of Terminalia chebula extract; and
100 to 500 mg of palm fruit extract.

2. The composition of claim 1, comprising, in every 1000 mg, 3 to 8 mg of astaxanthin.

3. The composition of claim 1, wherein the astaxanthin comprises 20% to 100% of left-handed astaxanthin.

4. The composition of claim 1, comprising, in every 1000 mg, 50 to 100 mg of seaweed calcium composition.

5. The composition of claim 1, wherein the seaweed calcium composition comprises 10 to 50 wt % of calcium.

6. The composition of claim 1, wherein the seaweed calcium composition further comprises at least one micronutrition, which is selected from the group consisting of Magnesium, phosphorous, sulphur, iron, boron, fluoride, sodium, cobalt, copper, zinc, selenium, molybdenum, iodine, manganese, and nickel.

7. The composition of claim 1, wherein the seaweed calcium composition comprises a honeycomb-like porous structure.

8. The composition of claim 1, wherein the calcium of the seaweed calcium composition comprises is of a polymorph, comprising 60% to 70% of calcite, 20% to 30% of aragonite, and 10% to 20% of vaterite.

9. The composition of claim 1, comprising, in every 1000 mg, 20 to 60 mg of collagen.

10. The composition of claim 1, wherein the collagen is undenatured Collagen Type II.

11. The composition of claim 1, comprising, in every 1000 mg, 50 to 100 mg of hyaluronic acid.

12. The composition of claim 1, comprising, in every 1000 mg, 10 to 30 mg of calcium fructoborate.

13. The composition of claim 1, comprising, in every 1000 mg, 200 to 300 mg of Terminalia chebula extract.

14. The composition of claim 1, wherein the Terminalia chebula extract comprises ellagitannin, chebulinic acid, hydrolyzable tannin, or a combination thereof.

15. The composition of claim 1, wherein the Terminalia chebula extract is obtained by a method, comprising: obtaining a peel of a Terminalia chebula fruit, grinding the peel to obtain a powder, extracting the powder using water to obtain a Terminalia chebula peel water-soluble extract, and drying the Terminalia chebula peel water-soluble extract to obtain a powder-form extract.

16. The composition of claim 1, comprising, in every 1000 mg, 200 to 400 mg of palm fruit extract.

17. The composition of claim 1, wherein the palm fruit extract comprises palmitoylethanolamide.

18. The composition of claim 1, comprising, in every 1000 mg,
about 4 mg of astaxanthin;
about 95 mg of seaweed calcium composition, comprising 10 to 50 wt % of calcium;
about 40 mg of undenatured collagen Type II;
about 80 mg of hyaluronic acid;
about 25 mg of calcium fructoborate;
about 250 mg of Terminalia chebula extract; and
about 350 mg of palm fruit extract.

19. The composition of claim 1, wherein the arthritis is degenerative arthritis.

20. The composition of claim 1, being an oral composition.
